(19) United States
(12) Patent Application Publication
(10) Pub. No.: US 2004/0077715 A1
(43) Pub. Date: Apr. 22, 2004

(54) Use of an isothiocyanate, a thiocyanate or a mixture thereof as depigmenting agent

(76) Inventor: Daniel Jean, Vic-Le-Comte (FR)

Correspondence Address: D Douglas Price, Steptoe & Johnson, 1330 Connecticut Avenue NW, Washington, DC, 20036, US

(21) Appl. No.: 10/470,079

(22) PCT Filed: Jan. 24, 2002
(86) PCT No.: PCT/FR02/00288

(30) Foreign Application Priority Data
Jan. 26, 2001 (FR) 01/01078

(51) Int. Cl. A61K 31/26; A61K 7/00

(52) U.S. Cl. 514/514; 424/401

(57) ABSTRACT

The invention concerns the use if isothiocyanate of general formula (I): R₁—N═C═S wherein: R₁ represents an alkyl, alkenyl, alkynyl, aryl, acetyl, alkylcarbonyl, alkoxy, cycloalkyl, aryloxy, arylcarbonyl group, carboxylic acid or a —(CH₂)ₙR₃ group wherein n represents an integer ranging between 1 and 5 and R₃ represents a polar functional group, advantageously a halogen atom, a sulphoxide, carbonyl, nitro, thioester, thioether, sulphonyl, sulphinyl, nitrile group, carboxylic acid, carboxylic ester, alkylthio or hydroxyl, a thiocyanate of general formula (II): R₂—S═C═N wherein: R₂ represents an alkyl, alkenyl, alkynyl, aryl, acctyl, alkylcarbonyl, alkoxy, cycloalkyl, aryloxy, arylcarbonyl group, carboxylic acid, carboxylic ester or a —(CH₂)ₙR₃ group, wherein n represents an integer ranging between 1 and 5 and R₃ represents a polar functional group, advantageously a halogen atom, a sulphoxide, carbonyl, nitro, thioester, thioether, sulphonyl, sulphinyl, nitrile group, carboxylic acid, carboxylic ester, alkylthio or hydroxyl, or mixtures thereof for making a medicine or a cosmetic composition for inhibiting tyrosinase.

The present invention relates to depigmenting agents and in particular to the use of isothiocyanates or thiocyanates as depigmenting agent.

The pigmentation of the skin in human beings originates from a complex series of cell processes which takes place in a single population of cells known as melanocytes. The melanocytes are situated in the lower part of the epidermis and their function is to synthesize a brown pigment, melanin, which protects the body from the damaging effects of ultraviolet radiation. The melanin is deposited in the melanosomes, vesicles present inside the melanocytes. The melanosomes are expelled from the melanocytes and conveyed towards the surface of skin by the keratinocytes, which assimilate the melanin present in the melanosomes. The dark complexion of the skin is proportional to the amount of melanin synthesized by the melanocytes and transferred to the keratinocytes. In some cases, it is preferable to reduce or inhibit melanogenesis, for example to lighten the skin, to remove blemishes due to ageing or to reduce hyperactivity of the melanocytes.

Cosmetic compositions comprising a peroxide, such as hydrogen peroxide or zinc peroxide, have been used for a long time with the aim of removing blemishes, such as freckles, which appear on the skin. However, peroxides are extremely unstable and consequently are problematic to store. Furthermore, the stable incorporation of these peroxides in cosmetic bases is difficult and peroxides themselves do not have a sufficiently whitening effect.

On the other hand, cosmetic preparations comprising vitamin C, cysteine or colloidal sulphur have begun to be used for the purpose of whitening the skin. However, the effects of these substances are not satisfactory.

Hydroquinone has for a long time been the reference depigmenting molecule employed in numerous dermocosmetic preparations. However, this product is not without danger and exhibits a significant cytotoxicity towards the melanocytes which is capable of bringing about irreversible depigmentation.

Kojic acid has recently been effectively used as substance for inhibiting the formation of melanin in the human skin. Consequently, various cosmetic preparations intended to depigment the skin and which comprise kojic acid (Japanese Patent Publication No. 56-18569) or an ester of kojic acid with an aromatic carboxylic acid, such as cinnamic acid or benzoic acid (Japanese Patent Publication No. 60/100005), or diester of kojic acid (Japanese Patent Publications Nos. 61-60801 and 60-17961) have been disclosed. These kojic acids and kojic acid esters are therefore known as being substances capable of inhibiting melanogenesis. However, kojic acid has an effectiveness which can vary from one individual to another and which, on average, is insufficient.

Consequently, the search for other depigmenting products is still of topical interest.

Surprisingly, the Applicants have discovered that certain molecules belonging to the family of the thiocyanates and isothiocyanates have a very marked inhibiting effect on tyrosinase in vitro.

The isothiocyanates can be extracted from various cruciferous species, including broccoli, Lepidium dabra and radishes, such as sulforaphane and sulforaphen.

Sulforaphane and some of its synthetic analogues are known to protect against the mutagenic effect of chemical substances, such as, for example, those present in tobacco smoke. This effect involves the induction of enzymatic systems involved in the discharge of the mutagenic molecules from the body. It would also appear that these molecules also act directly on the mechanism of mutagenesis (WO 94/19948, Carcinogenesis, 8, 12, 1987, pages 1971-1973; Cancer Research, 51, 13, 1991, pages 2063-2068).

However, the action of these substances as depigmenting agent has never been described.

The present invention thus relates to the use of an isothiocyanate of following general formula I:

R₁—N═C═S I

in which R₁ represents an alkyl, alkenyl, alkynyl, aryl, acetyl, alkylcarbonyl, alkoxy, cycloalkyl, aryloxy, arylcarbonyl, carboxylic acid or carboxylic ester group or a —(CH₂)ₙR₃ group in which n represents an integer ranging from 1 to 5 and R₃ represents a polar functional group, advantageously a halogen atom or a sulphoxide, carbonyl, nitro, thioester, thioether, sulphonyl, sulphinyl, nitrile, carboxylic acid, carboxylic ester, alkylthio or hydroxyl group, of a thiocyanate of following general formula II:

R₂—S═C═N II

in which R₂ represents an alkyl, alkenyl, alkynyl, aryl, acetyl, alkylcarbonyl, alkoxy, cycloalkyl, aryloxy, arylcarbonyl, carboxylic acid or carboxylic ester group or a —(CH₂)ₙR₃ group in which n represents an integer ranging from 1 to 5 and R₃ represents a polar functional group, advantageously a halogen atom or a sulphoxide, carbonyl, nitro, thioester, thioether, sulphonyl, sulphinyl, nitrile, carboxylic acid, carboxylic ester, alkylthio or hydroxyl group, or of their mixtures in the manufacture of a medicament or of a cosmetic composition intended to inhibit tyrosinase.

The term “alkyl group” is understood to mean, within the meaning of the present invention, any substituted or unsubstituted and linear or branched alkyl group comprising 1 to 10 carbon atoms, in particular the CH₃ group.

The term “alkenyl group” is understood to mean, within the meaning of the present invention, any substituted or unsubstituted and linear or branched alkenyl group comprising 2 to 10 carbon atoms, in particular the vinyl group.

The term “alkynyl group” is understood to mean, within the meaning of the present invention, any substituted or unsubstituted and linear or branched alkynyl group comprising 2 to 10 carbon atoms, in particular the ethynyl group.

The term “alkylcarbonyl group” is understood to mean, within the meaning of the present invention, any alkyl group as defined above bonded via a carbonyl group. An alkylcarbonyl group example is the acetyl group.

The term “alkoxy group” is understood to mean, within the meaning of the present invention, any substituted or unsubstituted and linear or branched alkoxy group comprising 1 to 10 carbon atoms, in particular the OCH₃ group.

The term “cycloalkyl group” is understood to mean, within the meaning of the present invention, any ring composed of alkyl group comprising 1 to 10 carbon atoms which is or is not substituted, in particular the cyclohexyl group.

The term “aryl group” is understood to mean, within the meaning of the present invention, one or more aromatic rings having 5 to 8 carbon atoms which can be joined or fused and substituted or unsubstituted. In particular, the aryl groups can be phenyl or naphthyl groups and the substituents can be halogen atoms, alkoxy groups as defined above, alkyl groups as defined above, or the nitro group.

The term “aryloxy group” is understood to mean, within the meaning of the present invention, an aryl group as defined above bonded via an alkoxy group as defined above.

The term “aralkyl group” is understood to mean, within the meaning of the present invention, any aryl group as defined above bonded via an alkyl group as defined above. In particular, an aralkyl group is a benzyl group.

The term “arylcarbonyl group” is understood to mean, within the meaning of the present invention, any aryl group as defined above bonded via a carbonyl group. An arylcarbonyl group example is the benzoyl group.

The term “carboxylic acid” is understood to mean, within the meaning of the present invention, any alkyl group as defined above to which a carboxyl group (—COOH) is bonded.

The term “sulphonyl group” is understood to mean, within the meaning of the present invention, any alkyl, cycloalkyl or aryl group as defined above bonded via an SO₂ group.

The term “sulphinyl group” is understood to mean, within the meaning of the present invention, any alkyl, cycloalkyl or aryl group as defined above bonded via an SO group.

The term “alkylthio group” is understood to mean, within the meaning of the present invention, any alkyl group as defined above bonded via a sulphur atom.

The present invention also relates to the use of an isothiocyanate of general formula I, of a thiocyanate of general formula II or of their mixtures in the manufacture of a medicament or of a cosmetic composition intended to lighten or depigment the epidermis or to remove blemishes due to ageing.

The thiocyanate is advantageously a thiocyanate of general formula II in which R₂ represents the aralkyl group; more advantageously still, it is benzyl thiocyanate.

The thiocyanate of general formula II is advantageously in the form of a salt, more advantageously still in the form of a sodium or potassium salt.

The thiocyanates can be obtained at the same time as the isothiocyanates during the decomposition of the glucosinolates of the cruciferous species by myrosinase (Pharmacognosie, Phytochimie, Plantes Médicinales [Pharmacognosy, Phytochemistry, Medicinal Plants], Bruneton, published by Lavoisier, Paris, 1993, p. 177). Some are synthetic and are available commercially, such as benzyl thiocyanate, from Fluka (ref. 13929).

In a specific embodiment, the isothiocyanate of formula I is a synthetic isothiocyanate, in particular in which R₁ represents an aryl, acetyl, alkylcarbonyl, cycloalkyl, arylcarbonyl or arylalkyl group. The isothiocyanate is advantageously chosen from the group consisting of cyclohexyl isothiocyanate, benzyl isothiocyanate, acetyl isothiocyanate and benzoyl isothiocyanate.

The synthetic isothiocyanates are available commercially. Thus, cyclohexyl isothiocyanate, benzyl isothiocyanate and benzoyl isothiocyanate are available from Aldrich (ref. C10-540-6, 25,249-2 and 26,165-3 respectively) and acetyl isothiocyanate from Fluka (ref. 01230).

The other isothiocyanates can be synthesized according to the method and examples indicated in U.S Pat. No. 5,411,986.

In another specific embodiment, the isothiocyanate of general formula I is obtained by extraction of a cruciferous species advantageously chosen from the group consisting of broccoli, Lepidium dabra and radishes. More advantageously still, it is chosen from the group consisting of sulforaphane and sulforaphen.

In particular, the process of extraction of the cruciferous species comprises the following stages:

Treatment of the cruciferous species, advantageously lyophilized, with a water-miscible solvent or a water/solvent mixture, advantageously acetone,

Concentration of the solution obtained, advantageously under reduced pressure,

Filtration of the product obtained,

Treatment with silver nitrate at 0°C.,

Filtration of the argentic complex precipitate thus formed,

Displacement of this complex with sodium thiosulphate,

Extraction of the suspension obtained with a water-immiscible organic solvent advantageously chosen from the group consisting of chloroform, ether, ethyl acetate and their mixture, more advantageously still an ethyl ether/chloroform mixture,

Drying of the organic phase,

Optionally purification of the product obtained, in particular by thin-layer chromatography.

The following examples of the preparation of sulforaphane and sulforaphen by extraction of cruciferous species are given by way of indication without implied limitation.

### EXAMPLE 1

### Preparation of Sulforaphane

90 g of lyophilized broccoli (Brassica oleracea italica) are treated with three decoctions at reflux in 75% acetone.

The extractive solutions are combined and concentrated under reduced pressure to 100 g. The concentrate is filtered through a filter paper. The filtrate is brought to 0°C. and 100 ml of a 60% aqueous silver nitrate solution are added. It is filtered the precipitate through a sintered glass filter and is rinsed with three times 100 ml of distilled water. The precipitate is subsequently treated with 100 ml of a 60% aqueous sodium thiosulphate solution, which is allowed to act at 0°C. with stirring for two hours.

The suspension obtained is subsequently extracted in a separating funnel with six times 50 ml of an ethyl ether/chloroform (8/2 v/v) mixture. The organic phase is dried over sodium sulphate and then evaporated under reduced pressure. 32 mg of crude sulforaphane are obtained. The residue is deposited on a silica gel preparative chromatography plate and elution is carried out with a mixture of isopropanol and methanol (7/3 v/v).

The plate is visualized with ammoniacal silver nitrate over a small portion, in order to determine the region of migration of the sulforaphane. This region is scraped off and the sulforaphane is extracted from the silica with chloroform. The chloroform is evaporated and 9 mg of sulforaphane are obtained. The sulforaphane is identified by gas chromatography coupled to a mass spectrometer.

### Example 2

### Preparation of Sulforaphen

The preparation is carried out in the same way as on broccoli but using radish seeds (Raphanus sativus).

7 mg of sulforaphen are obtained after purification by thin-layer chromatography.

### Example 3

### Synthesis of (D,L)-Sulforaphane

40 g of 4-chlorobutyronitrile (ref. Aldrich C 3,000-0) are dissolved in 800 ml of absolute ethyl alcohol distilled beforehand over sodium.

27 g of methanethioate (ref. Fluka 71742) are subsequently added and the mixture is left stirring at 25° C. for 15 hours. The suspension is filtered through a filter paper and the filtrate is evaporated under reduced pressure. The residue is taken up in 400 ml ethyl ether. Filtration is again carried out through a filter paper. An ethereal solution comprising 32 g of crude 4-methylthiobutyronitrile is obtained.

A suspension of 25 g of lithium aluminium hydride in 400 ml of ethyl ether is prepared.

The 4-methylthiobutyronitrile solution is gradually added to the lithium aluminium hydride suspension and then the mixture is brought to reflux for 2 h 30.

The suspension is subsequently neutralized by slowly adding, under reflux, 80 ml of distilled water. When boiling has ceased, 120 ml of distilled water are subsequently added to bring the neutralization of the remaining hydride to completion. The mixture is filtered through a sintered glass funnel. The insoluble material is washed on the filter with 200 ml of ethyl ether. The ethereal fractions are combined and evaporated to dryness. 26.9 g of methylthiobutylamine are obtained. The product obtained is taken up in 80 ml of acetone, to which 23 ml of 35% hydrogen peroxide are gradually added. The mixture is placed overnight on a water bath at 50° C.

A small amount of active charcoal is subsequently added, the mixture is filtered and 200 ml of chloroform comprising 20 ml of thiophosgene are slowly added, followed by 300 ml of a 5% aqueous sodium hydroxide solution. Reaction is allowed to take place for 30 min.

The mixture is subsequently extracted countercurrent wise with 8 times 200 ml of dichloromethane. The organic phase is collected, dried over sodium sulphate and evaporated.

The residue is subsequently rectified at 135° C. under 7×10⁻² torr. 12.5 g of D,L-sulforaphane are obtained, the identity of which is confirmed by mass spectrometry.

The following examples of the measurement of the power to inhibit tyrosinase are given by way of indication without implied limitation.

### Measurement of the Power to Inhibit Tyrosinase

The following reaction is used: colourless L-Dopa (L-3,4-dihydroxyphenylalanine, obtained from Sigma (ref. D-9628)) is oxidized to coloured dopachrome, which absorbs at 475 nm. This reaction is catalyzed by fungal tyrosinase (EC. 1.14.18.1, obtained from Sigma (ref. T-7755)). The kinetics of the reaction are recorded by the measurement of the optical density (O.D.) as a function of the time at 30° C.

The compositions of the various solutions used are as follows:

| | |
|---|---|
| Monopotassium phosphate | 0.70 g |
| Disodium phosphate | 0.69 g |
| Distilled water | q.s. 100 ml |

### Substrate Solution

L-Dopa at 0.35% (w/v) in the pH 6.5 buffer solution.

### Solutions of Inhibitors

The inhibiting molecules are dissolved directly in the pH 6.5 buffer, in 50% methanol (methanol/distilled water) or in pure methanol, depending upon their solubility.

The concentrations in weight by volume of the various solutions of inhibitors are: 0.2%, 0.1%, 0.05%, 0.025%, 0.0125%, 0.00625% and 0.00312%.

### Enzyme Solution

Tyrosinase at 0.010% (w/v) in the pH 6.5 buffer solution.

The amounts of these various solutions used during the tests are presented in Tables 1 and 2 for each reaction studied:

TABLE 1
Enzyme-substrate reaction

| | Blank | Test |
|---|---|---|
| Buffer | 1.8 ml | 1.3 ml |
| Substrate | 1 ml | 1 ml |
| Enzyme | 0 ml | 0.5 ml |
| Solvent of the inhibitor | 0.2 ml | 0.2 ml |
| Inhibitor | 0 ml | 0 ml |

TABLE 2
Enzyme-substrate-inhibitor reaction

| | Blank | Test |
|---|---|---|
| Buffer | 1.8 ml | 1.3 ml |
| Substrate | 1 ml | 1 ml |
| Enzyme | 0 ml | 0.5 ml |
| Solvent of the inhibitor | 0.2 ml | 0 ml |
| Inhibitor at various | 0 ml | 0.2 ml |
| concentrations | | |

The action of tyrosinase is evaluated by the initial rate of the reaction measured on the O.D. recording.

The initial rates of reactions without inhibitors (concentration 0) and the rates at the various concentrations tested are plotted on a curve.

The inhibiting power of a molecule is defined as the concentration which reduces the action of tyrosinase by 50%.

The molecules tested as inhibitor were acquired from Aldrich or Fluka depending upon the products, with the exception of sulforaphane and sulforaphen, prepared in the way indicated in Examples 1 and 2.

The results obtained on the molecules tested are collated in the following table:

| | Concentration which inhibits |
|---|---|
| | the enzymatic activity of |
| | tyrosinase by 50%, in % |
| Molecule | (weight/volume) |
| Hydroquinone (Aldrich | 0.154 |
| ref. 24,012-5) (reference | |
| molecule) | |
| Cyclohexyl isothiocyanate | 0.104 |
| (Aldrich ref. C10-540-6) | |
| Benzyl isothiocyanate | 0.0980 |
| (Aldrich ref. 25,249-2) | |
| Sulforaphane | 0.103 |
| Sulforaphen | 0.112 |
| Acetyl isothiocyanate | 0.055 |
| (Fluka ref. 01230) | |
| Benzoyl isothiocyanate | 0.0063 |
| (Aldrich ref. 26,165-3) | |
| Potassium thiocyanate | 0.20 |
| Benzyl thiocyanate (Fluka | 0.25 |
| ref. 13929) | |

Sulphoraphane inhibits tyrosinase approximately 1.5 times more than hydroquinone.

It is thus observed that all the isothiocyanates used in the table are superior to hydroquinone and that the most active of them, benzoyl isothiocyanate, is approximately 24 times more active than hydroquinone.

The thiocyanates, for their part, have an activity similar to that of hydroquinone.

### Test of the Depigmenting Power of Benzoyl Isothiocyanate and of Sulforaphane in Comparison With Kojic Acid and with Hydroquinone on Pigmented Guinea Pigs

Guinea pigs with a pigmented skin, shaved beforehand, received twice daily, 5 days out of 7, during one to two months of treatment, applications of a glycerol-based cream comprising 5% of kojic acid (ref. Aldrich 22,046-9) or 5% of hydroquinone (ref. Aldrich H 1,790-2) or 5% of benzoyl isothiocyanate (ref. Aldrich 30,818-8) or 5% of synthetic sulforaphane prepared according to Example 3. These applications were carried out on circular regions with a diameter of 2 cm pinpointed by indelible marking using yellow ink.

After treating for 4 weeks and after a period of desquamation of the skin at the hydroquinone and benzoyl isothiocyanate spots, a significant whitening of the skin is recorded for all the products, except for kojic acid, which has no effect.

### Test of the Power of Sulforaphane, in Comparison With Kojic Acid, to Inhibit the Synthesis of Melanin With Regard to Cultured Melanocytes

Hydroquinone cannot be used as reference product in this test because of its excessively high cytotoxicity.

These tests were carried out on different batches of cells during experiments which were independent and repeated (8 times).

The synthesis of melanin kinetically or after treatment for 6 days (once daily) was tested; the viability of the cells is confirmed by MTT and/or by staining with crystal violet.

The results are expressed in mg/ml of melanin in so far as the cells are inoculated at the same concentration.

At a concentration of 0.00035%, hydroquinone and sulforaphane inhibit the synthesis of melanin, whereas kojic acid and benzoyl isothiocyanate no longer have an effect. Over all of the tests (8 independent tests), the following results were obtained at a concentration of 0.00035% for all the products: 26.22% inhibition for sulforaphane, 1% for kojic acid, 9.5% for benzoyl isothiocyanate and 42% for hydroquinone.

### Test of the Power to Inhibit the Synthesis of Melanin With Regard to Pigmented Human Skin Reconstituted in Culture

Type-6 pigmented epidermis (corresponding to black skin), in a proportion of three samples per test, were treated daily for 5 days with a control cream with the following composition (as % by weight):

| | |
|---|---|
| Cetearyl alcohol | 7 |
| Sodium cetearyl sulphate | 0.7 |
| Stearic acid | 3 |
| Glycerol | 20 |
| Mixture of nipa esters in phenoxyethanol | 0.5 |
| Triethanolamine | 0.2 |
| Distilled water | 68 |

with kojic acid at a concentration of 3.5×10⁻⁵ (w/w) and with sulforaphane at a concentration of 3.5×10⁻⁶ (w/w) in the same cream acting as excipient.

At the end of the treatment, the culture skin is left for a further two days and the melanin is extracted from the treated skin and from the control skin with a mixture of solvents and sodium hydroxide (Solvable® from Packard Bioscience B.V.) and the melanin extracted is quantified by colorimetry according to a method described previously (Chemical Characterization of Hair Melanins in Various Coat-Color Mutants of Mice; Hiroyuki Ozeki et al., J. Invest. Dermatol. 105, 3, 1995, 361-366).

It is found that kojic acid inhibits the synthesis of melatonin by 8% and sulforaphane by 30%, although its concentration is ten times lower than that of kojic acid.

1. Use of an isothiocyanate of following general formula I:
R₁—N═C═S I
in which R₁ represents an alkyl, alkenyl, alkynyl, aryl, acetyl, alkylcarbonyl, alkoxy, cycloalkyl, aryloxy, arylcarbonyl, carboxylic acid or carboxylic ester group or a —(CH₂)ₙR₃ group in which n represents an integer ranging from 1 to 5 and R₃ represents a polar functional group, advantageously a halogen atom or a sulphoxide, carbonyl, nitro, thioester, thioether, sulphonyl, sulphinyl, nitrile, carboxylic acid, carboxylic ester, alkylthio or hydroxyl group, of a thiocyanate of following general formula II:
R₂—S═C═N II
in which R₂ represents an alkyl, alkenyl, alkynyl, aryl, acetyl, alkylcarbonyl, alkoxy, cycloalkyl, aryloxy, arylcarbonyl, carboxylic acid or carboxylic ester group or a —(CH₂)ₙR₃ group in which n represents an integer ranging from 1 to 5 and R₃ represents a polar functional group, advantageously a halogen atom or a sulphoxide, carbonyl, nitro, thioester, thioether, sulphonyl, sulphinyl, nitrile, carboxylic acid, carboxylic ester, alkylthio or hydroxyl group, or of their mixtures in the manufacture of a medicament or of a cosmetic composition intended to inhibit tyrosinase.

2. Use according to claim 1, characterized in that the thiocyanate of general formula II is in the form of a salt, advantageously in the form of a sodium or potassium salt.

3. Use according to claim 1, characterized in that the isothiocyanate of general formula I is obtained by extraction of a cruciferous species advantageously chosen from the group consisting of broccoli, Lepidium dabra and radishes.

4. Use according to claim 3, characterized in that the isothiocyanate is chosen from the group consisting of sulforaphane and sulforaphen.

5. Use according to claim 1, characterized in that the isothiocyanate of general formula I is a synthetic isothiocyanate chosen from the group consisting of cyclohexyl isothiocyanate, benzyl isothiocyanate, acetyl isothiocyanate and benzoyl isothiocyanate.

6. Use according to one of the preceding claims in the manufacture of a medicament or of a cosmetic composition intended to lighten or depigment the epidermis or to remove blemishes due to ageing.

7. A depigmenting or lightening medicament or cosmetic composition, comprising an isothiocyanate of formula I:
R₁—N═C═S (I)
in which R₁ represents an alkyl, alkenyl, alkynyl, aryl, acetyl, alkylcarbonyl, alkoxy, cycloalkyl, aryloxy, arylcarbonyl, carboxylic acid or carboxylic ester group or a —(CH₂)ₙR₃ group in which n represents an integer from 1 to 5 and R₃ represents a polar functional group, a thiocyanate of formula II:
R₂—S═C═N (II)
in which R₂ represents an alkyl, alkenyl, alkynyl, aryl, acetyl, alkylcarbonyl, alkoxy, cycloalkyl, aryloxy, arylcarbonyl, carboxylic acid or carboxylic ester group or a —(CH₂)ₙR₃ group in which n represents an integer from 1 to 5 and R₃ represents a polar functional group, or a mixture thereof.

8. The composition according to claim 7, wherein the polar functional groups are a halogen atom or a sulphoxide, carbonyl, nitro, thioester, thioether, sulphonyl, sulphinyl, nitrile, carboxylic acid, carboxylic ester, alkylthio or hydroxyl group.

9. The composition according to claim 7, wherein the thiocyanate of formula II is in the form of a salt.
